(19) United States
(12) Patent Application Publication
(10) Pub. No.: US 2016/0101108 A1
(43) Pub. Date: Apr. 14, 2016

(54) PRINTED DOSAGE FORMS

(71) Applicant: ÅBO AKADEMI UNIVERSITY, Turku (FI)

(72) Inventors: Niklas SANDLER, Turku (FI); Natalja GENINA, Turku (FI); Jouko PELTONEN, Turku (FI); Petri IHALAINEN, Turku (FI)

(73) Assignee: Åbo Akademi University, Turku (FI)

(21) Appl. No.: 14/892,997

(22) PCT Filed: May 23, 2014
(86) PCT No.: PCT/FI2014/050403

§ 371 Date: Nov. 20, 2015

(30) Foreign Application Priority Data
May 23, 2013 (FI) 20135555

(51) Int. Cl. A61K 31/525; A61K 33/10; A61K 9/20; A61K 31/375; A61K 31/465; A61K 31/455; A61K 33/08

(52) U.S. Cl. CPC A61K 31/525; A61K 31/455; A61K 33/10; A61K 33/08; A61K 31/375; A61K 31/465; A61K 9/2095

(57) ABSTRACT

The present invention relates to oral dosage forms of vitamin(s) and/or dietary mineral(s) or nicotine produced by printing techniques. The present invention also relates to a method of producing an oral dosage form of vitamin(s) and/or dietary mineral(s) or nicotine by printing technique(s).

### FIELD OF THE INVENTION

The present invention relates to oral dosage forms of vitamin(s) and/or dietary mineral(s) produced by a printing technique. The present invention relates also to oral dosage forms for nicotine replacement therapy produced by a printing technique. The present invention also relates to a method of producing an oral dosage form of vitamin(s) and/or dietary mineral(s) or an oral dosage form for nicotine replacement therapy by printing technique(s).

### BACKGROUND OF THE INVENTION

Oral solid dosage forms of medicines as well as vitamin(s) and/or dietary mineral(s) are most often made into tablets or capsules. The drug substance and the additives are processed through multiple production steps including crystallization and powder milling of the drug substance or the dietary supplement, addition of the additives, mixing the ingredients, granulating, tableting and coating of the final tablets, for example.

In drug manufacturing, the control of the solid-state properties of drug substances is essential and it offers opportunities for enhancement of drug delivery systems. In this context, inkjet printing technologies offer solutions for controlling material and product characteristics with high precision.

Inkjet printing is found to provide the ability to obtain uniformly spaced and sized droplets on surfaces to create precision patterns of the ink. Inkjet techniques allow printing on various surfaces. The concept of inkjet printing technology for printing pharmaceutical dosage forms of paracetamol, caffeine and theophylline on porous substrates (uncoated paper, coated paper and polyethylene terephthalate film) has been studied in J Pharm Sci., 2011; 100(8):3386-3395. In addition, printing technologies have been used to produce drug delivery systems for riboflavin sodium phosphate and propranolol hydrochloride (Eur. J of Pharm. Sci. (2012), 47, 3, 615-623).

Inkjet printing technology has been suggested to offer new perspectives for solving problems related to poorly soluble drugs and dosing low-dose medicines accurately.

### BRIEF DESCRIPTION OF THE INVENTION

The invention is based on the observation that inkjet printing technology can be used to manufacture printed dosage forms of vitamin(s) and/or dietary mineral(s) on carrier substrates. In addition, the invention is based on the observation that inkjet printing technology can be used to tailor individualized multivitamin and dietary supplement preparations for patients/customers. These observations can be used as a basis for personalized and/or optimized administration of certain selected vitamins and/or dietary minerals to patients/customers. In addition, the current invention provides a novel and effective means for optimizing the dose(s) of vitamin(s) and/or dietary mineral(s) in a dietary supplement composition by using a printing technology.

The invention is also based on the observation that inkjet printing technology can be used to manufacture printed dosage forms for nicotine replacement therapy. In addition, the invention is based on the observation that inkjet printing technology can be used to tailor individualized nicotine replacement preparations for patients/customers. These observations can be used as a basis for personalized and/or optimized administration of nicotine for patients needing help with smoking cessation.

Accordingly, an object of the present invention is to provide a printed oral dosage form comprising dietary supplement(s), such as vitamin(s) and/or dietary mineral(s), wherein at least one vitamin and/or dietary mineral is included in the substrate material and at least one vitamin and/or dietary mineral is printed on the substrate. A further object of the present invention is to provide a printed oral dosage form for nicotine replacement therapy. Another object of the present invention is to provide a method for manufacturing a printed oral dosage form of vitamin(s) and/or dietary supplement (s) or nicotine. A further object of the present invention is to provide a printed oral dosage form of vitamins, dietary supplements or nicotine, wherein the components and/or the doses of the components in the dosage form are personalized and/or optimized based on the need of an individual. In addition, an object of the present invention is to provide a method of personalizing a printable oral dosage form of dietary supplements or nicotine based on the need of an individual. An even further object of the present invention is to provide a method for optimizing the doses of vitamin(s) and/or dietary mineral(s) or nicotine in a composition based on the need of an individual by manufacturing the composition by a printing technology. The need of the individual is determined by diagnosed deficiencies of certain vitamin(s), mineral(s) and/or nicotine in the individual, by the type and/or strength of the withdrawal symptoms in the individual or by the weight of the individual, for example.

The objects of the invention are achieved by the products, methods and uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows a schematic presentation of the concept of one embodiment of the present invention.

FIG. 2 shows a series of the printed dosage forms comprising increasing doses of vitamin B2.

FIG. 3 shows a nicotine release curve.

### DETAILED DESCRIPTION OF THE INVENTION

The present invention is based on the finding that inkjet printing technology can be used in manufacturing dietary supplement and multivitamin preparations and in individualising the combinations and/or doses of vitamin(s) and/or dietary mineral(s) in multivitamin and dietary supplement products. This observation can be used as a basis for both the individualized dosage forms of vitamins and/or dietary minerals and the personalized administration of vitamins and/or dietary minerals. The invention is also based on the finding that inkjet printing technology can be used in manufacturing nicotine replacement therapy preparations and in individualising the doses of nicotine in nicotine replacement therapy products. This observation can be used as a basis for both the individualized dosage forms of nicotine and the personalized administration of nicotine, especially for patients needing help with their smoking cessation.

Vitamins and dietary minerals are nutrients that are considered as essential for maintaining and/or promoting health of an individual.

Vitamins are divided into fat soluble vitamins, such as A-, D-, E-, and K-vitamins and water soluble vitamins, such as C- and the B-vitamins. Vitamins, in general, have multiple functions in a human. Vitamin A is important, for example, for growth and development, for the maintenance of the immune system and good vision. Vitamin E has many biological functions, the antioxidant function being the most important. Human body needs vitamin K for post-translational modification of certain proteins required for blood coagulation, and in metabolic pathways in bone, for example. B-vitamins play important roles in cell metabolism. Table 1 lists the vitamins and their typical amounts in multivitamin and/or dietary supplement preparations.

TABLE 1
Vitamins and their typical amounts in multivitamin
and/or dietary supplement preparations

| Ingredient | Typical amount in multivitamins, mg |
|---|---|
| Vitamin A (retinol) | 0-0.5 |
| Vitamin B (thiamine) | 1-5 |
| Vitamin B2 (riboflavin) | 1-5 |
| Vitamin B3 (niacin) | 10-25 |
| Vitamin B5 (pantothenic acid) | 4-6 |
| Vitamin B6 (pyridoxine) | 2-4 |
| Vitamin B9 (folic acid) | 0.1-0.3 |
| Vitamin B12 (cobalamin) | 0.001-0.01 |
| Vitamin C (ascorbic acid) | 50-150 |
| Vitamin D3 (cholecalciferol) | 0.01-0.02 |
| Vitamin E (tocopherol) | 5-15 |
| Vitamin K (quinones) | 0-0.05 |

Dietary minerals such as manganese, zinc, iron, copper, iodine, selenium and molybdenum, which are also called dietary supplements, are needed to support the vital functions in a human. Manganese is a cofactor in enzyme functions. Zinc is pervasive and required for several enzymes such as carboxypeptidase, liver alcohol dehydrogenase and carbonic anhydrase. Iron is required for many proteins and enzymes, notably hemoglobin to prevent anemia. Copper is required component of many redox enzymes, including cytochrome C oxidase. Iodine is required for the synthesis of thyroid hormones, and selenium is a cofactor essential to activity of antioxidant enzymes like glutathione peroxidase. Table 2 lists the dietary minerals and their typical amounts in multivitamin and/or dietary supplement preparations.

TABLE 2
Dietary minerals and their typical amounts in multivitamin
and/or dietary supplement preparations

| Ingredient | Typical amount in multivitamins, mg |
|---|---|
| Magnesium | 0-200 |
| Calcium | 0-120 |
| Zinc | 0-15 |
| Chrome | 0-50 |
| Iron | 0-5 |
| Copper | 0-1 |
| Selene | 0-0.05 |

Nicotine replacement therapy (NRT) is the remedial administration of nicotine to the body as part of smoking cessation. The products approved for NRT release nicotine in a form that does not involve the risks of smoking. Typical forms of administration of nicotine in the NRT are patches and gums, which administer nicotine transdermally and transmucosally/orally, respectively. NTR is meant to be used for a short period of time and should optimally be tapered down to a low dose before stopping. The primary benefit of nicotine replacement therapy is that it prevents craving for tobacco in a smoker and thus enables the easier overcoming of nicotine addiction. The use of NRT increases the chances of stopping smoking by 50 to 70% compared to placebo or to no treatment.

Printing technologies, such as inkjet printing, flexographic printing, 3D-printing, screen printing as well as liquid dispensing and pipetting methods offer possibilities to deposit a variety of materials onto different types of carrier materials or substrates, and can be used in the manufacture of the printed dosage forms of the present invention. Inkjet printing has the ability to dispense uniform droplets in the picoliter range with high degree of accuracy. Here, however, the ink solution has to be formulated to have viscosity and surface tension to guarantee continuous printing and reproducibility of the droplets. In flexographic printing, on the other hand, different types of ink solutions can be printed onto a wide variety of substrates. Flexographic printing lacks dosing precision and every ink must be printed separately, which make printing of pharmaceutical dosage forms and/or multicomponent systems problematic. By combining the inkjet printing and the flexographic printing, the advantages of high dosing precision and flexibility could be achieved.

There are two major types of inkjet technologies, namely continuous and drop-on-demand, which can be adapted to print several materials. The properties of the printed fluids have a crucial role in successful printing. The vitamin(s) and/or mineral(s) are dissolved in a solution having optimal properties for printability, such as viscosity and surface tension. Suitable viscosity, for example, in the range of 2-20 cP, and surface tension, for example, in the range of 25-65 mN/m, are essential for controlled printing. Propylene glycol, glycerol and/or PEG, for example, can be used in adjusting the viscosity of the solution. Other properties for the ink solution is that vitamin(s) and/or mineral(s) and possible additives are dissolved or are present in small particle sizes, typically less than 1 μm. Too low viscous ink solution would penetrate the nozzles by themselves and fluid with too high viscosity might not exit the nozzle at all. The surface tension has to be adjusted to enable the formation of a round drop after the detachment from the nozzle. The viscosity and surface tension values and requirements depend greatly on the technical properties of the printer. Further, the jetting parameters, such as firing voltage, drop spacing and volume, for example, are aspects which can be used in controlling the characteristics of the printed formulation.

The present invention relates to printed dosage forms or formulations of different categories of vitamins and/or dietary minerals. The present invention relates also to printed dosage forms or formulations of nicotine. The printed dosage form of the present invention comprises a carrier material, a substrate, and at least one vitamin and/or mineral, or nicotine deposited on the substrate by printing. In one embodiment, the invention relates to a printed oral dosage form of nicotine or vitamin(s) and/or dietary mineral(s). In another embodiment, the invention relates to a printed oral dosage form of vitamin(s) and/or dietary mineral(s), wherein the dosage form comprises at least two of vitamin(s) and/or mineral(s) and at least one vitamin and/or dietary mineral is included in the substrate material and at least one vitamin and/or dietary mineral is printed on the substrate. In certain embodiment, the at least one vitamin and/or dietary mineral is included in the substrate material before printing the at least one other vitamin and/or dietary mineral on the substrate. In another embodiment, the dosage form comprises at least two vitamins or at least two dietary minerals and at least one vitamin and/or dietary mineral is included in the substrate material. In a further embodiment, the dosage form comprises at least one vitamin and at least one dietary mineral and at least one vitamin and/or dietary mineral is included in the substrate material. In an even further embodiment the dosage form comprises at least two vitamins and at least two dietary minerals and at least one vitamin and/or dietary mineral is included in the substrate material. In an even further embodiment, at least one vitamin and at least one dietary mineral are included in the substrate material. In one embodiment, the vitamin(s) and/or mineral(s) included in the substrate material is a high-dose vitamin and/or mineral. The vitamin(s) and/or mineral(s) can be formulated into the substrate material by any suitable method known in the art, such as powder compressing, powder compacting, impregnating or by exploiting flexographic printing or screen printing, for example. In one embodiment, the vitamin(s) included in the substrate material is selected from vitamin B3, vitamin C and/or vitamin E. In one embodiment, the mineral included in the substrate material is selected from magnesium, calcium, zinc and or chrome. In one embodiment, vitamin C, calcium, zinc and/or magnesium are included in the substrate. In a further embodiment, vitamin C, calcium and magnesium are included in the substrate. In one embodiment, the printed dosage form of the present invention comprises vitamin C, vitamin B2, vitamin B3, calcium and magnesium.

In one embodiment, the invention relates to a printed dosage form or a formulation of nicotine. In another embodiment, the invention relates to a printed oral or mucosal dosage form or a formulation of nicotine. In a certain embodiment, the invention relates to a printed oral dosage form of nicotine, wherein the dosage form comprises also a taste masking ingredient. In a further embodiment the invention relates to a printed oral dosage form of nicotine, wherein both nicotine and the taste masking ingredient are printed on the substrate. In an even further embodiment, the invention relates to a printed oral dosage form of nicotine, wherein the taste masking ingredient is included in the substrate material and nicotine is printed on the substrate. In a certain embodiment, the taste masking ingredient is included in the substrate material before printing nicotine on the substrate.

The taste masking ingredient can be formulated into the substrate material by any suitable method known in the art, such as powder compressing, powder compacting, impregnating or by exploiting flexographic printing or screen printing, for example.

The taste masking ingredient can be selected from ingredients typically used in NRT-products, such as, flavoring agents like mint, liquorice and fruit flavors, complexation compounds, sugars and/or extracts, for example.

The present invention relates also to a method for manufacturing a printed oral dosage form of least one vitamin and/or dietary mineral or nicotine, wherein the method comprises the steps of

a) providing a substrate or a carrier material, optionally comprising at least one vitamin and/or dietary mineral or a taste masking ingredient,

b) providing at least one ink solution comprising vitamin(s) and/or dietary mineral(s) or nicotine,

c) printing the ink solution(s) on the substrate,

d) optionally adding protecting and/or functional layer(s) onto the printed dosage form produced in the step (c),

e) recovering the final printed dosage from.

In one embodiment of the present invention, inkjet printing is used in depositing the ink solution on the substrate.

Further, the present invention relates to use of a printing technique for manufacturing an oral dosage form comprising at least two of vitamin(s) and/or dietary minerals or nicotine. In one embodiment, inkjet printing is used as the printing technique for depositing the ink solution on the substrate.

Additionally, the present invention relates to a method of personalizing a printable oral dosage form of vitamin(s) and/or mineral(s), by determining the vitamin and/or dietary mineral deficiencies of an individual, and manufacturing the dosage form containing the vitamins and/or minerals needed by the individual by printing the dosage form. Further, the present invention relates to a method for optimizing the dose of vitamin(s) and/or dietary mineral(s) in a dosage form by determining the required vitamin and/or dietary mineral doses for an individual, and manufacturing the dosage form containing the vitamins and/or minerals in the required doses by a printing technology.

Further, the present invention relates to a method of personalizing a printed oral dosage form of nicotine by determining the required dose of nicotine of an individual, and manufacturing the dosage form containing the amount of nicotine needed by the individual by a printing technology.

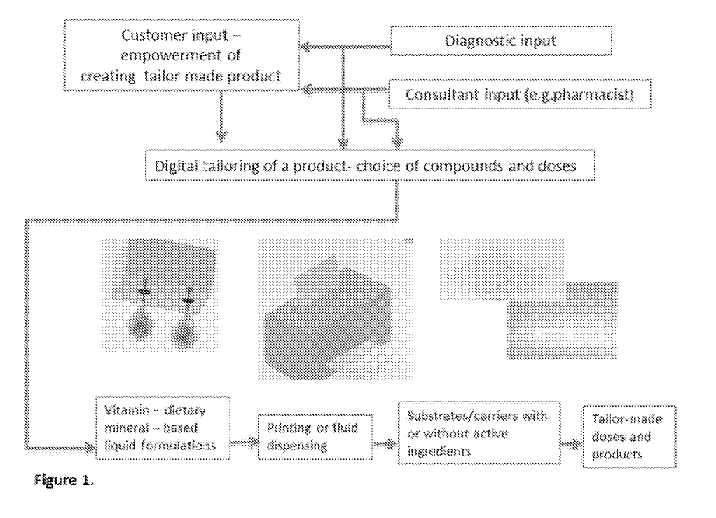

The vitamin(s) and/or dietary mineral(s) in the printed dosage form or to be printed to the dosage form can be selected or personalized based on the need of an individual. In one embodiment, the vitamin(s) and/or dietary mineral(s) are selected or personalized based on diagnosed deficiencies of certain vitamin(s) and/or mineral(s) in an individual, on the type and/or strength of the deficiency symptoms in the individual, or on the weight of the individual. Further, the doses of vitamin(s) and/or dietary mineral(s) or nicotine in the printed dosage form can be optimized or personalized based on the need of an individual. In one embodiment, the doses of vitamin(s) and/or dietary mineral(s) are based on diagnosed deficiencies of certain vitamin(s) and/or mineral(s) in an individual, on the type and/or strength of the deficiency symptoms in the individual, or on the weight of the individual. In one embodiment, the deficiency of certain vitamin(s) and/or mineral(s) in an individual is diagnosed by a laboratory test or by typical symptoms associated with the deficiency in question. In another embodiment, the dose of nicotine in the printed dosage form is determined based on the diagnosed deficiency or by the type and/or strength of the withdrawal symptoms in the individual. A schematic presentation of the concept of one embodiment of the present invention is shown in FIG. 1.

Vitamins and/or dietary minerals and nicotine are typically dissolved in a solvent solution, ink solution, having optimal properties, such as surface tension and viscosity, for printability. The vitamins and/or dietary minerals are dissolved in an aqueous solution or an organic solvent or mixtures thereof depending on the properties of the vitamin and/or dietary mineral. In one embodiment, the vitamin(s) and/or dietary mineral (s) are dissolved in water. In another embodiment, the vitamin(s) and/or dietary mineral(s) are dissolved in water/ethanol mixture. The vitamin(s) and/or dietary mineral(s) containing ink solution may also comprise edible mineral or vegetable oils and/or lipids, especially when fat-soluble ingredients are involved. The vitamin(s) and/or dietary mineral(s) containing ink solution comprises optionally also viscosity modifier(s) and/or moisturizers, such as propylene glycol, glycerol, polyethylene glycol (PEG) and/or sodium carboxymethyl cellulose, for example. Also surface active agents, such as ethanol, isopropanol and surfactants, may be used In one embodiment, the solvent solution is propylene glycol/water mixture. In another embodiment, the solution is propylene glycol/water 30:70 mixture (vol-%). In one embodiment, the solution is a mixture of glycerol, ethanol and water. In another embodiment, the solution is glycerol/ethanol/water 10:10:80 mixture (vol-%). Further, suspensions, colloidal or nano-particulate solutions and various formulation approaches, common for example in the pharmaceutical field, to enhance the properties of inherently poorly soluble substances, such as, A-, D-, E- and K-vitamins, can be used. Vitamin(s) and/or dietary mineral(s) containing ink solution can comprise one vitamin and/or one dietary mineral or it can comprise two or more vitamin(s) and/or dietary mineral(s). In one embodiment, each vitamin or dietary mineral is included in its own ink solution. In another embodiment, the ink solution comprises at least two vitamins, at least two dietary minerals or at least one vitamin and at least one dietary mineral.

The substrate or carrier material can be chosen from materials that are able to host the printed compounds, i.e., the vitamins and/or minerals or nicotine in their structures. Further, the substrate material can be chosen from materials that are able to release the vitamins, dietary minerals and nicotine slowly, sustainably or quickly, depending on the characteristics and/or the intended use of the preparation in question. The properties of the substrate material that contribute to the control of the printed substances are porosity, surface energy, surface roughness, capillary action and chemical components on the surface, for example. In one embodiment of the present invention, the substrate material is porous material. In another embodiment, the substrate material is nonporous material. Cellulosic materials and other biodegradable materials, such as starch- and rice-based materials can be used as a substrate according to the present invention. In one embodiment, the substrate material is sugar based decor paper or rice paper. In one embodiment, the substrate material is edible material. In another embodiment the substrate material does not dissolve in the human digestive system. In another embodiment, the substrate material is any kind of orodispersible material or film. In one embodiment, the substrate material is a chitosan film or a hydroxypropyl methylcellulose film. In a further embodiment, the substrate material is suitable to be administered orally or buccally. In one embodiment, the substrate material is pharmaceutically acceptable material. In another embodiment, the substrate material is recognized by the relevant authorities to be safe in food applications. The substrate can contain also flavor compounds which are deposited on the material by printing or by other techniques.

After deposition of the vitamin(s) and/or dietary mineral(s) or nicotine containing ink solution on the substrate by printing, the printed patterns can be coated by one or more additional protecting or functional layers to produce the final dosage form. The coating can be a film made of a water soluble polymeric material or a water insoluble polymeric material. The thickness of the coating is typically from few hundred nanometres to few hundred micrometres. The printed patterns can be coated using the techniques know and available in the pharmaceutical and/or food industry, such as spray coating, spin coating, flexographic printing or inkjet printing. In one embodiment of the invention, flexographic printing, inkjet printing or screen printing is used for coating. The dosage forms can be coated by printing one or more additional protecting or functional layers on the top of the initial vitamin and/or mineral supplement dose.

According to the present invention, the printing techniques enable manufacturing on-demand individualized multivitamin or dietary supplement preparations containing the vitamin(s) and/or mineral(s) the customer needs at personalized doses. The printing techniques thus enable manufacturing on-demand multivitamin or dietary supplement preparations comprising vitamin A which is absent from many multivitamin preparations due to concerns over overdosing or use during pregnancy. In addition, printing techniques enable manufacturing on-demand individualized multivitamin or dietary supplement preparation containing vitamin K, which is absent from many multivitamin preparations due to the need to adjust warfarin dosage. Further, printing techniques enable manufacturing multivitamin or dietary supplement preparations which are tailored with regard to the seasonal needs of vitamin(s), such as vitamin D. Additionally, printing techniques enable manufacturing on-demand personalized multivitamin or dietary supplement preparations based on individual diagnosed deficiencies of certain vitamin(s) and/or mineral(s). Further, printing techniques enable manufacturing on-demand personalized multivitamin or dietary supplement preparations in cases where the doses of the dietary supplements depend upon the weight of the individual, such as children and the elderly. In addition, printing techniques enable manufacturing on-demand personalized multivitamin or dietary supplement preparations for individuals having difficulties or reluctance in swallowing. The printing techniques enable also manufacturing on-demand personalized multivitamin or dietary supplement preparations in hospital environment wherein hospital pharmacy can manufacture individual preparations based on the laboratory results and/or diagnoses. Further, the printing techniques enable manufacturing of dosage forms including in addition to vitamin(s) and/or mineral(s) also pharmaceutically active ingredient(s) for which the vitamin(s) and/or mineral(s) is essential, for example. In addition, the printing techniques enable manufacturing on-demand individualized nicotine preparations containing nicotine at personalized doses. The printing techniques enable also manufacturing on-demand personalized nicotine preparations for nicotine replacement therapy in pharmacy environment, for example, wherein the pharmacy can manufacture preparations containing individual doses of nicotine based on the laboratory results and/or the type and/or strength of the withdrawal symptoms in the individual.

In one embodiment of the invention, the printed dosage forms are arranged to a series of multiple dosage forms. In one embodiment, the dose of at least one of the vitamins and/or minerals increases. In another embodiment, the dose of at least one of the vitamins and/or minerals or nicotine decreases within the series. In a certain embodiment, the dose of at least one vitamin, such as vitamin B2 or vitamin B3, increases or decreases within the series.

The increasing dosing of vitamin(s) and/or mineral(s) in the series of multiple dosage forms is determined by the diagnosed vitamin and/or dietary mineral deficiencies of an individual. The decreasing dosing of nicotine, vitamin(s) and/or mineral(s) in the series of multiple dosage forms is determined by the laboratory results and/or the type and/or strength of the nicotine withdrawal symptoms in the individual or by the diagnosed vitamin and/or dietary mineral deficiencies of an individual, for example.

Further, the present invention relates to a method of personalizing a printed oral dosage form of nicotine by determining the required dose of nicotine of an individual, and manufacturing the dosage form containing the amount of nicotine needed by the individual by a printing technology.

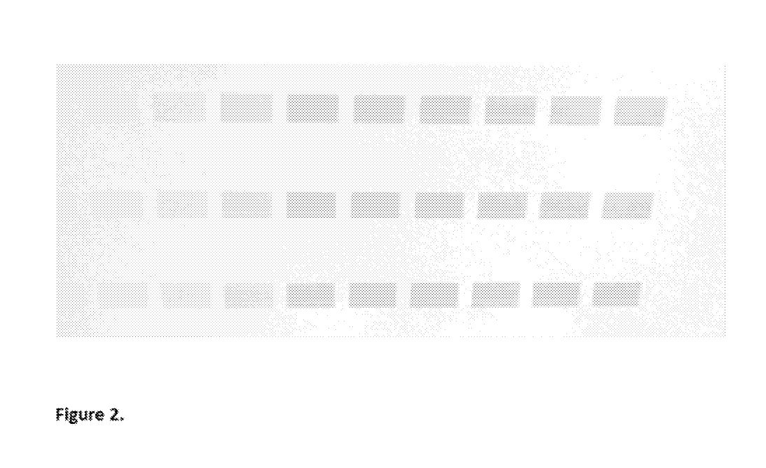

A series of the printed dosage forms comprising increasing doses of vitamin B2 is shown in FIG. 2.

Additionally, the present invention relates to a use of the printed dosage form or the series of multiple dosage forms according to the present invention for treating a vitamin and/or dietary mineral deficiency in an individual or for a nicotine replacement therapy in an individual.

The invention will be described in more detail by means of the following examples. The examples are not to be construed to limit the claims in any manner whatsoever.

### Example 1

### Printed Formulations of Vitamin B2 and Vitamin B3

Two different ink solutions were used to print the vitamins onto the substrates. The ink solution containing 50 mg/ml of niacin (vitamin B3) was produced by dissolving the vitamin in propylene glycol/water (30/70 vol-%) mixture. The ink solution containing riboflavin sodium phosphate (vitamin B2) was produced by dissolving the vitamin in glycerol/ethanol/water (10/10/80 vol-%) mixture. The solutions were filtered with 0.2 μm polypropylene membrane filter (Whatman, GE Healthcare, Piscataway, N.J., USA) before printing.

The substrate materials used were normal copy paper and multilayer curtain coated paper.

Inkjet printing was performed with a Dimatix DMP-2800 inkjet printer (Fujifilm Dimatix Inc. Santa Clara, Calif., USA). In the printer, a MEMS-based cartridge-styled printhead with 16 nozzles linearly spaced at 254 μm that produce a nominal drop size of 10 μl was used. Printing was performed in ambient conditions (relative humidity 45.5±5%, 21±1° C.) with a single nozzle using firing voltage of 35 V. The cartridge temperature was 30° C., and the drops were deposited at a drop spacing of 10 μm, 20 μm and 30 μm. The vitamins were printed in squares of 1 cm×1 cm on normal copy paper (n=6) and on multilayer curtain coated paper (n=1).

The theoretical doses mg/area for vitamin B3 were 0.501, 0.125 and 0.056, respectively. The theoretical doses mg/area for vitamin B2 were 0.32, 0.08 and 0.035.

Table 3 shows the information of the flexible doses.

TABLE 3
Flexible doses

| Vitamins | Vitamin B3 | | | Vitamin B2 | | |
|---|---|---|---|---|---|---|
| Drop spacing: | 10 | 20 | 30 | 10 | 20 | 30 |
| Copy paper | n = 6 | n = 6 | n = 6 | n = 6 | n = 6 | n = 6 |
| Multilayer paper | n = 1 | n = 1 | n = 1 | n = 1 | n = 1 | n = 1 |
| Printing area | 1 | 1 | 1 | 1 | 1 | 1 |
| (cm²): | | | | | | |
| Dose (mg)/area | 0.501 | 0.125 | 0.056 | 0.32 | 0.08 | 0.035 |
| (theoretical): | | | | | | |

The actual doses of vitamin B2 and vitamin B3 on the printed areas were analyzed. Each printed area was immersed into predetermined amount of water and vigorously shaken. The absorbance values of the obtained solutions were measured with UV/Vis-spectrophotometer at 262 nm and 267 nm (373 nm) for vitamin B3 (niacin) and vitamin B2 (RSP), respectively after 4 h and 24 h of incubation. The calibration curves were done in water, as well.

These results show that inkjet printing can be used to produce uniform solid dosage forms once the conditions affecting the printing are calibrated.

Tables 4 and 5 show the results for vitamin B3 and Vitamin B2, respectively.

TABLE 4
Vitamin B3 doses

| 4 h | mg | | |
|---|---|---|---|
| DS | theoretical | measured | SD |
| 10 | 0.501 | 0.7594 | 0.0059 |
| 20 | 0.125 | 0.2213 | 0.0040 |
| 30 | 0.056 | 0.0717 | 0.0008 |

| | mg | |
|---|---|---|
| 24 h | | 262 nm |

| DS | theoretical | measured | SD |
|---|---|---|---|
| 10 | 0.501 | 0.7323 | 0.0052 |
| 20 | 0.125 | 0.2142 | 0.0029 |
| 30 | 0.056 | 0.0687 | 0.0012 |

TABLE 5
Vitamin B2 doses

| | mg | | | | |
|---|---|---|---|---|---|
| 4 h | | 267 nm | | 373 nm | |
| DS | theoretical | measured | SD | measured | SD |
| 10 | 0.32 | 0.6255 | 0.0055 | 0.6459 | 0.0066 |
| 20 | 0.08 | 0.1337 | 0.0063 | 0.1403 | 0.0081 |
| 30 | 0.035 | 0.0623 | 0.0021 | 0.0658 | 0.0019 |
| 10 | 0.32 | 0.5847 | 0.0108 | 0.6141 | 0.0111 |
| 20 | 0.08 | 0.1199 | 0.0081 | 0.1276 | 0.0107 |
| 30 | 0.035 | 0.0521 | 0.0018 | 0.0560 | 0.0016 |

### Example 2

### Substrate Comprising at Least One Vitamin and/or Dietary Mineral

The substrate was made by powder compression of 250 mg calcium carbonate (CaCO₃), 83 mg magnesium oxide (MgO) and 60 mg vitamin C.

The amounts of vitamin(s) and/or dietary mineral(s) in substrate were 100 mg of Ca, 50 mg of Mg, and 60 mg vitamin C.

By inkjet printing vitamin B2 and vitamin B3 can then be printed on the substrate to produce a printed dosage form comprising vitamin C, vitamin B2, vitamin B3, calcium and magnesium.

### Example 3

### Printed Formulation of Nicotine

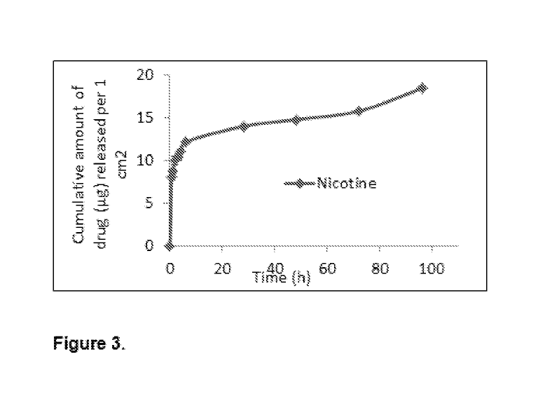

Nicotine hydrogen tartrate solution (16.6 mg/ml) in PDMS was used in producing the printed formulation. A nicotine release curve of a flexographically prepared sample (PDMS-nicotine) is shown in FIG. 3.

The number of droplets deposited can be accurately controlled (one droplet in the this setup being 15-20 picoliters) allowing tunable nicotine replacement therapy products to be produced flexibly based on patient or customer feedback, counselling by health care professionals, diagnostic testing or other decision making approach.

1-25. (canceled)

26. A printed oral dosage form of vitamin(s) and/or dietary mineral(s), comprising at least two of vitamin(s) and/or mineral(s), wherein at least one vitamin and/or dietary mineral is included in the substrate material and at least one vitamin and/or dietary mineral is printed on the substrate.

27. The printed oral dosage form according to claim 26, wherein the dosage form comprises at least two vitamins or at least two dietary minerals.

28. The printed oral dosage form according to claim 26, wherein the dosage form comprises at least one vitamin and at least one dietary mineral.

29. The printed oral dosage form according to claim 26, wherein the vitamin(s) and/or mineral(s) in the composition are personalized to an individual based on diagnosed deficiencies of certain vitamin(s) and/or mineral(s) in the individual.

30. The printed oral dosage form according to claim 26, wherein the doses of the vitamin(s) and/or mineral(s) in the composition are personalized to an individual based on diagnosed deficiencies of certain vitamin(s) and/or mineral(s) in the individual.

31. The printed oral dosage form according to claim 29, wherein the deficiencies of vitamin(s) and/or dietary mineral(s) and/or the required doses of vitamin(s) and/or dietary mineral(s) are diagnosed by a laboratory test or by typical symptoms associated with the deficiency in question.

32. A series of the printed oral dosage form according to claim 26.

33. The series according to claim 32, wherein the dose of at least one of the vitamins and/or minerals increases, or the dose of at least one of the vitamins and/or minerals decreases within the series.

34. A method for manufacturing a printed oral dosage form of a vitamin and/or mineral supplement, wherein the method comprises the steps of
a) providing a substrate or a carrier material, optionally comprising at least one vitamin and/or dietary mineral or a taste masking ingredient,
b) providing at least one ink solution comprising vitamin(s) and/or dietary mineral(s),
c) printing the ink solution(s) on the substrate,
d) optionally adding protecting and/or functional layer(s) onto the printed dosage form produced in step (c),
e) recovering the final printed dosage from.

35. A method of personalizing a printed oral dosage form of vitamin(s) and/or mineral(s) comprising at least one vitamin and/or dietary mineral included in the substrate material and at least one vitamin and/or dietary mineral printed on the substrate, wherein the method comprises the steps of determining the vitamin and/or dietary mineral deficiencies of an individual, and manufacturing the dosage form containing the vitamins and/or minerals needed by the individual by a printing technology.

36. A method for optimizing the dose of vitamin(s) and/or dietary mineral(s) in a dosage form comprising at least one vitamin and/or dietary mineral included in the substrate material and at least one vitamin and/or dietary mineral printed on the substrate wherein the method comprises the steps of determining the required vitamin and/or dietary mineral doses of an individual, and manufacturing the dosage form containing the vitamins and/or minerals in the required doses by a printing technology.

37. The method according to claim 35, wherein the deficiencies of vitamin(s) and/or dietary mineral(s) and/or the required doses of vitamin(s) and/or dietary mineral(s) are diagnosed by a laboratory test or by typical symptoms associated with the deficiency in question or by the type and/or strength of the withdrawal symptoms in the individual.
